(19) United States
(12) Patent Application Publication
(10) Pub. No.: US 2003/0092062 A1
(43) Pub. Date: May 15, 2003

(54) Immobilizing biological molecules

(76) Inventors: M. Parameswara Reddy, Brea, CA (US); Firdous Farooqui, Brea, CA (US)

Correspondence Address: BECKMAN COULTER INC, 4300 NORTH HARBOR BOULEVARD, P O BOX 3100, FULLERTON, CA, 928343100

(21) Appl. No.: 10/033,308

(22) Filed: Oct. 24, 2001

(51) Int. Cl. G01N 33/53; C08G 63/48; C08G 63/91; B05D 3/00

(52) U.S. Cl. 435/7.1; 427/2.11; 525/54.5

(57) ABSTRACT

This invention provides a system for immobilizing biological molecules onto a solid support having an available amino group that uses two steps. In a first step, a nucleophilic substitution reaction occurs so that the available amino group displaces a first leaving group of an activating compound to form an activated support. In a second step, the activated support reacts with biological molecules resulting in a composition of the formula:

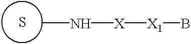

wherein S is a solid support, preferably X is selected from the group consisting of

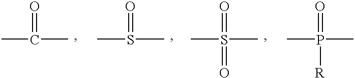

wherein R is selected from the group consisting of alkyl, aryl, and OR¹ having no greater than about 18 carbon atoms,

wherein R¹ is selected from the group consisting of alkyl and aryl having no greater than about 18 carbon atoms,

wherein X₁ is selected from the group consisting of NH, oxygen, and sulfur, and wherein B is a biological molecule.

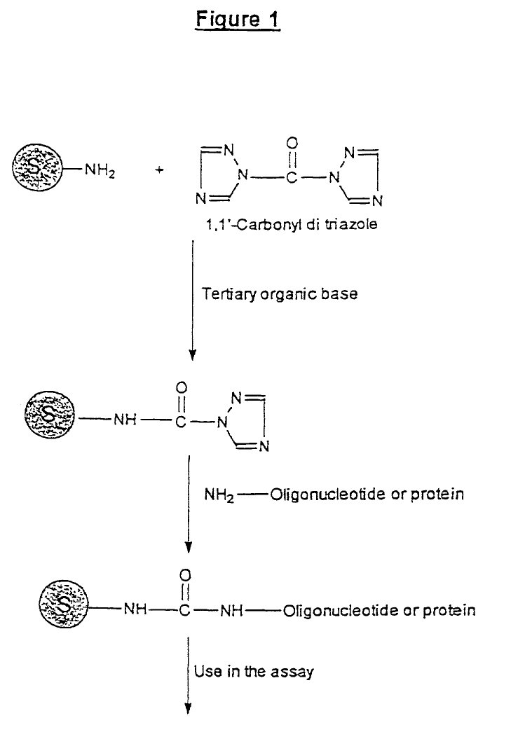

### BACKGROUND

The following description provides a summary of information relevant to the present invention and is not a concession that any of the information provided or publications referenced herein is prior art to the presently claimed invention.

Biological molecules immobilized or attached to solid supports are useful in diagnostic and analytical procedures, such as assays. One method for immobilizing biological molecules onto solid supports uses acyl fluoride (“AcF”) as an activating compound. Information relevant to this method of attachment can be found in U.S. Pat. Nos. 6,146,833 and 6,110,699. Another method uses carbonyl diimidazole (“CDI”) as an activating compound to attach biological molecules to a solid support. The steps for the activation method and coupling chemistry of the CDI method are described in Greg T. Hermanson, A. Krishna Mallia, and Paul K. Smith, “Immobilized Affinity Ligand Techniques,” pages 64-67 (1992 Academic Press Inc.

The two methods described above suffer from one or more disadvantages. The AcF method and CDI method have more than two steps, and additional steps increase the cost of attachment of a biological molecule. For example, these methods require additional linker chemistry, such as a succinylation step, before attachment of biological molecules occur in the method. As more steps are necessary to attach the biological molecule in the method, preparation costs increase. The AcF and CDI methods also result in a low loading of biological molecules onto a solid support. The AcF and CDI methods, as a consequence of this low loading, have diminished sensitivity when used in an assay for detection of a low concentration of an analyte.

Accordingly, a need exists for a system useful in immobilizing biological molecules to solid supports that is more efficient, more economical, simpler, and faster than the alternatives as well as providing greater sensitivity for analyte detection.

### SUMMARY

The present invention satisfies that need. The invention provides a method for attaching biological molecules with at least one reactive amino, thiol or hydroxyl group to a solid support having at least one available amino group.

A method according to the present invention comprises reacting: (i) a solid support with at least one available amino group and (ii) an activating compound.

Optionally the reaction is in a first solution, where the activating compound is soluble in the first solution. The activating compound has the structure:

L₁—X—L₂ (1)

wherein L₁ and L₂ are leaving groups, namely groups that can be displaced in nucleophilic substitution reactions, and X is a moiety capable of nucleophilic substitution. L₁ and L₂ can be independently selected from the group consisting of halogen, imidazole, triazole, pyrrole pyrazole, thiazole, tetrazole and O-Aryl-R,

wherein R is selected from a group consisting of halogen, nitro, cyano, and alkoxy moiety. Preferably, X is selected from the group consisting of:

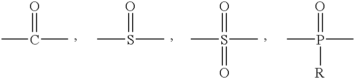

wherein R is selected from the group consisting of alkyl, aryl, and OR¹ having no greater than about 18 carbon atoms, and

wherein R¹ is selected from the group consisting of alkyl and aryl having no greater than about 18 carbon atoms.

The reaction in the first step results in L₁ being displaced by the available amino group on the solid support to form an activated support.

The second step of the method of the present invention comprises reacting a biological molecule having at least one reactive amino, thiol or hydroxyl group with the activated solid support. Optionally the second step occurs in a second solution. When a low concentration of a solution comprising the biological molecule in the second solution is deposited onto one or more sites of the activated support, preferably the reaction occurs in a humid chamber. The chemical reaction in the second step results in the reactive amino, thiol, or hydroxyl group of the biological molecule displacing L₂, and attaching the biological molecule to the solid support. The solid-support with an attached biological molecule has the following formula:

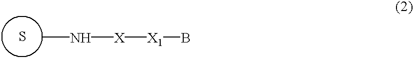

wherein X₁ is selected from the group consisting of NH, oxygen, and sulfur provided by the reactive amino, thiol, or hydroxy group respectively of the biological molecule, and

wherein B is the biological molecule.

### DRAWING

These features, aspects and advantages of the present invention will become better understood with regard to the following description, appended claims and accompanying drawing which shows the steps of the method of the present invention using 1,2,4-carbonyl di triazole as the activating compound.

### DESCRIPTION

The following discussion describes embodiments of the invention and several variations of these embodiments. This discussion should not be construed, however, as limiting the invention to these particular embodiments. Practitioners skilled in the art will recognize numerous other embodiments as well.

The invention provides a system for attaching biological molecules having at least one reactive amino, thiol or hydroxyl group to a solid support having at least one available amino group, and the novel compositions formed thereof. The invention has several advantages. One advantage is the invention is simpler, faster, and less costly than the CDI and AcF methods because it uses less steps to attach biological molecules to solid supports. Another advantage is the invention is more efficient, and results in higher loading of biological molecules onto solid supports than the AcF and CDI methods. Another advantage is the invention has shown greater sensitivity than the CDI method in detection of an analyte.

The invention is useful in preparation of components for analytical and diagnostic procedures, such as a component in an assay or drug detection kit. The invention can be used, for example, in an assay involving biological molecules such as for the quantity and presence of cytokines, the presence of a disease state in an organism, the quantity and presence of a therapeutic drug, and detection of nucleic acids resulting from underlying infections.

Biological molecule as referred to herein encompasses any organic molecule, and includes but is not limited to oligonucleotides, nucleic acids, such as DNA and RNA, polypeptides, haptens, and carbohydrates. Polypeptide as referred to herein encompasses, and includes but is not limited to proteins and antibodies, and any fragments thereof. Haptens are generally small molecules, such as drugs, hormones, and synthetic compounds including but not limited to compounds associated with the use of therapeutic drugs and drugs of abuse.

Examples of haptens associated with drugs of abuse include but are not limited to compounds associated with the metabolism or use of cocaine, morphine, and nicotine. Examples of haptens in terms of therapeutic drugs include but are not limited to compounds associated with the use of tobramycin, phenobarbitol, theophylline, digoxin, and gentamycin.

Biological molecules used in the method of the present invention have at least one reactive amino, thiol or hydroxyl group. Examples of biological molecules with at least one reactive amino, thiol or hydroxyl group include polypeptides with at least one surface amino group, amino derivatized oligonucleotides, thiolated oligonucleotides, and thiol containing proteins. Polypeptides, haptens, and carbohydrates with at least one reactive amino, thiol or hydroxyl group, can be purchased from Sigma, P.O. Box 14508, St. Louis, Mo. 63178. When the polypeptide is a protein or antibody with a three dimensional configuration, the location of the amino group or other reactive group on the surface of the molecule is preferable for the substitution reaction to occur, and to attach the polypeptide to the solid support. Numerous polypeptides with at least one surface reactive amino, thiol, or hydroxy group can be used in the present invention. For example, polypeptides with lysine as a component typically have at least one surface amino group. Lysine is an amino acid with an available amino group. Other polypeptides that can be used include proteins containing sulfhydroxy groups.

Example of oligonucleotides as the biological molecule having the reactive group include amino derivatized oligonucleotides and oligonucleotides having at least one free thiol group. Amino oligonucleotides can be synthesized on a 3′-Amino-Modifier C7 CPG (purchased from Glen Research, 22825 Davis Drive, Sterling, Va. 20164) following the manufacturer's protocol using a DNA synthesizer ABI 394 (purchased from Applied Biosystems, 850 Lincoln Centre Drive, Foster City, Calif. 94404). The amino group can be placed at 3′ end or at 5′ end of oligonucleotide. The 3′ amino oligonucleotide is preferably used in the preparation of amino oligonucleotides for the present invention. A method for preparing amino derivatized oligonucleotides is also described in U.S. Pat. No. 6,110,669 which is incorporated by reference hereto. In addition, oligonucleotides having at least one free thiol group can be synthesized on supports purchased from Glen Research following the manufacturer's protocol.

Those persons skilled in the art will recognize that biological molecules can be modified for use in assays, and other diagnostic or analytical procedures. The present invention contemplates these modifications.

With reference to the drawing, the present invention comprises two steps for immobilizing a biological molecule onto a solid support. The first step of the method comprises reacting in a first solution a solid support with at least one available amino group, and an activating compound soluble in the first solution to form an activated solid support. The second step of the method comprises reacting the activated solid support from the first step with a biological molecule having at least one reactive amino, thiol or hydroxyl group in a second solution. The second step results in the biological molecule attaching to the solid support.

Solid supports capable of having an available amino group attached thereto include a wide range of materials including but not limited to natural materials and synthetic materials. Natural materials include but are not limited to cellulose, and agarose. Synthetic materials include but are not limited to polypropylene, polystyrene, polymethacrylate, nylon. The solid supports used in the invention may take different forms such as a bead, plate, film, or other structures. Procedures for providing a solid support with an available amino group are well known in the art, and an example of a procedure is described in U.S. Pat. No. 5,112,736 which is incorporated by reference herein.

The first solution can be an organic solvent such as acteonitrile (“AcCN”), dimethyl formamide (“DMF”), dimethyl sulfoxide (“DMSO”), dichloromethane, dichloroethane, toluene, and tetrohydrofunan.

The activating compound has the structure:

L₁—X—L₂

wherein L₁ and L₂ are leaving groups in a nucleophilic substitution reaction, and X is a moiety capable of nucleophilic substitution. Preferably, L₁ and L₂ is independently selected from the group consisting of halogen, imidazole, triazole, pyrrole, pyrazole, thiazole, tetrazole, and O-Aryl-R, wherein R is selected from the group consisting of halogen, nitro, cyano, and alkoxy moiety. X is preferably selected from the group consisting of:

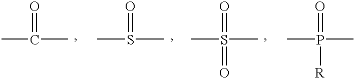

wherein R is selected from the group consisting of alkyl, aryl, and OR¹ having no greater than about 18 carbon atoms, and

wherein R¹ is selected from the group consisting of alkyl and aryl having no greater than about 18 carbon atoms.

A preferred activating compound is 1,2,4-carbonyl di triazole, which has the formula:

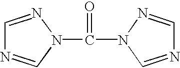

(1,2,4-carbonyl di-triazole is available from Sigma, St. Louis, Mo.) Other suitable activating compounds include but are not limited to carbonyl diimidazole (“CDI”), carbonyl dichloride, and nitrophenyl chloroformate.

Preferably, a tertiary organic base such as triethylamine, diisopropylamine, tributylamine, trimethylamine is added to the first solution to increase the rate and efficiency of the first reaction.

The chemical reaction that occurs in the first step is a nucleophilic substitution reaction between the activating compound and the available amino group on the solid support. A first leaving group, L₁ of the activating compound, becomes displaced by the available amino group on the solid support to form an activated support. When 1,2,4-carbonyl di triazole is the activating compound, the activated support has the following structure:

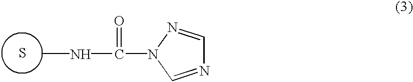

The second solution can be an aqueous solution containing a buffer, such as carbonate buffer, phosphate buffer, borate buffer, or can utilize an organic solvent such as DMF, DMSO, CH₃ CN, and CH₂Cl₂.

The chemical reaction that occurs in the second step is a nucleophilic substitution reaction between the activated solid support and the biological molecule in the second solution. The second leaving group, L₂ of the activating compound, becomes displaced by the reactive amino, thiol, or hydroxyl group of the respective biological molecule. The resulting novel composition has the following formula:

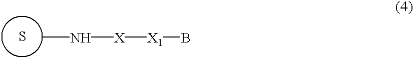

wherein X₁ is selected from the group consisting of NH, oxygen, and sulfur provided by the reactive amino, thiol, or hydroxy group respectively of the biological molecule, and

wherein B is the biological molecule.

When the activated support (3) reacts with an amino derivatized oligonucleotide, the resulting composition has the following structure:

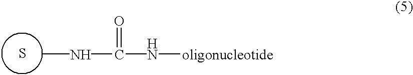

In another version of the present invention, the second step is preferably performed in a humid chamber when low concentrations of a solution comprising biological molecules in the second solution is deposited onto one or more sites of the activated solid support. Low concentration as used herein refers to a concentration of solution between about 5 to about 25 nanoliters within a circular spot having a diameter of between about 10 microns to about 500 microns. Preferably the humidity is at least 60 percent relative humidity. More preferably, the humidity in an enclosure forming the humid chamber is from about 80 to 100 percent relative humidity. When the low concentrations of the solution react in a humid chamber, a significantly higher loading of biological molecules onto the solid support occurs when compared against the same reactions in a non-humid chamber. Techniques to deposit or dispense low concentrations of solutions are often used in analytical or diagnostic procedures. For example, and not as a limitation, inkjet technology and piezo electric microjet printing technology have been used to deliver low concentrations of solution, and is referred to as “printing” spots on the solid supports. An array of spots having a low concentration of solution is printed onto the solid support. A method of printing biological molecules onto a solid support is described in U.S. Pat. No. 6,146,833 which is incorporated by reference herein.

The present invention is not limited to the preferred embodiments described in this section. The embodiments are merely exemplary, and one skilled in the art will recognize that many others are possible in accordance with this invention. Having now generally described the invention, the same will be more readily understood through references to the following examples, which are provide by way of illustration, and are not intended to be limiting of the present invention, unless so specified.

### EXAMPLE 1

### Preparation of Anine Derivatized Plates

This example describes the preparation of a solid support used in the invention. Polypropylene plates were aminated according to the procedure of U.S. Pat. No. 5,112,736 which is incorporated by reference herein. The plates were placed in plasma chamber and aminated with ammonia gas using a Plasma Science, Model 0150E Animator (Plasma Science, Airco Coating Technology, 2700 Maxwell Way, Fairfield, Calif. 94533) under following conditions:

Step I: Ammonia, 0.256 Torr, 4 min

Step II: Ammonia, 0.306 Torr, Plasma 40% power (RF), 2 min

Step III: Ammonia, 0.256 Torr, 2 min

Step IV: Ar, 0.265 Torr, 10 min

### EXAMPLE 2

### Comparison of Amino Oligonucleotide Loading onto Plates Between the Present Invention, CDI Method, and ACF Method in a Humid Chamber and Non-Humid Chamber

This example compared the present invention, CDI method, and AcF method in both a humid chamber and non-humid chamber. Aminated plates were prepared for each method according to Example 1. To create an activated solid support in accordance with the present invention, the animated plates were reacted with 1,2,4-carbonyl di-triazole (Sigma, St. Louis, Mo.) 0.1 M solution in anhydrous AcCN with 3-5% dry triethylamine in a glove box under argon for 2-3 hours. The plates were washed three times and air dried.

To create an activated solid support for the AcF method, the procedure described in U.S. Pat. Nos. 6,146,833 and 6,110,69 was used to create an activated solid support for the CDI method, the procedure described in Greg T. Hermanson, A. Krishna Mallia, and Paul K. Smith, “Immobilized Affinity Ligand Techniques,” pages 64-67 (1992 Academic Press Inc.) was used.

The biological molecules attached to the activated solid support for each method were 3′-amino oligonucleotide-5′-Cy3 synthesized on a 3′-Amino-Modifier C7 CPG (Glen Research, Sterling, Va.) following manufacturer's protocol on DNA synthesizer ABI 394 (Applied Biosystems, Foster City, Calif.). Cy3 is a cyanine dye which is a flurophore, and can be purchased from GlenReserach, 44901 Falcon Place, Sterling, Va. 20166. In each method, 20 ΦM of 3′-amino oligonucleotide-5′-Cy3 in bicarbonate buffer pH 9.3 with 4% Na₂SO₄ were deposited in about 5-25 nanoliters within a circular spot having a diameter of between about 10 microns to about 500 microns onto several sites of the plates in the form of 3×3 array by printing in a closed, dust free, and humid chamber using with a Biomek 2000 (Beckman Coulter, Fullerton, Calif.). The printed plates were left overnight in a humid chamber for one aspect of the experiment and a non humid chamber for the other aspect of the experiment. In each method, the unreacted active groups were quenched with 50 mM carbonate buffer, 150 mM NaCl, 1 mg/ml casein overnight at room temperature, washed with water, and imaged under a charge coupled device camera (“CCD camera”). The CCD camera collected fluorescent emission at 570 nm, and the intensity or brightness of the fluorescent emissions correlated to loading of the oligonucleotides onto the plates.

The CCD camera results for Example 2 showed that the present invention exhibited more intense signals, i.e. higher loading of oligonucleotides on the solid support, at each concentration level when compared against the other methods under their respective humid chamber and non-humid chamber conditions. Under the humid chamber conditions, the present invention exhibited greater intensity at every concentration than the other methods. Under the non-humid chamber conditions, the present invention exhibited a relative large increase in intensity with increase in concentration; whereas, the other methods exhibited a smaller increase in intensity as concentration increased.

The CCD camera results from Example 2 further showed that all methods, not merely the present invention, resulted in higher loading of the amino oligonucleotides when reacted in a humid chamber in contrast to the same results for the method in a non-humid chamber.

### EXAMPLE 3

### The Sensitivity of the Present Invention in Comparison to the CDI Method Using an IL 8 Assay

Example 3 describes the experimental protocol and results obtained from a comparison of the sensitivity of the present invention and the CDI method in detection of an analyte. Aminated plates were prepared for each method according to Example 1. 3′-amino oligonucleotide were made and printed according to the procedure discussed under Example 2 with two exceptions: The first exception was that the amino oligonucleotides printed in 3×3 arrays had 4 over prints so that the printing pin with liquid containing the amino oligonucleotides touched the surface of the plates five times. The second exception was the reactions took place only in the humid chamber.

An IL 8 assay was used to provide a comparative evaluation between the present invention and the CDI method. The protocol for the IL 8 assay used in this experiment was the same protocol discussed in U.S. Pat. No. 5,548,213 which is incorporated by reference herein. In particular, the protocol is described in detail below.

Step I. Added 140 ng/well of antibody-oligonucleotide conjugate in casein buffer and shook the plate at 37° C. for 1 hour. Washed the plate with wash buffer (0.02% Tween 20 in 1×Tris Buffer Saline) 3 times.

Step II. Added antigen ranging from 1000-1 pg/ml per well in casein buffer and reacted at 37° C. for 1 hour and washed with wash buffer 3 times. The concentrations of antigen used in the assay were: 0 pg/ml, 1 pg/ml, 5 pg/ml, 10 pg/ml, 25 pg/ml, 50 pg/ml, 250 pg/ml, and 1000 pg/ml.

Step III. Added biotinylated antibody (purchased from R & Ds systems, 614 McKinley Place N.E. Minnapolis, Minn. 55413) 50 ng per well and incubate at 37° C. for 1 hour, washed 3 times.

Step IV. Added Streptavidin PBx1 (purchased from Martek, 6480 Dobbin Road, Columbia, Md. 21045) 1 mg dissolved in 1 ml of water) 1:150 dilution, 50 Φ1/well and incubated at 37° C. for 1 hour and washed 3 times with wash buffer. 50 Φ1 of wash buffer were kept in each well and image using CCD camera. The CCD camera collected the fluorescent emission at 670 nm that correlated to loading of an oligonucleotide tagged antibody onto the solid support.

The CCD camera results from Example 3 showed that the present invention exhibited higher sensitivity for detection of an anlayte than the CDI method at the same concentrations of the analyte. The intensity of the fluorescent emissions or brightness of the spots correlated to the detection of the analyte. The present invention showed spots (brightness captured by CCD camera) starting at 1 pg/ml, and increasing in brightness as concentration increased to 1000 pg/ml. In contrast, the CDI method did not begin to show any spots until about 25 pg/ml. Thus, the present invention exhibited greater sensitivity than the CDI method

Not to be bound by theory, the inventors believed that the higher sensitivity of the present invention may be a result of higher loading of amino oligonucleotides. The higher loading obtained with the method of the present invention is presumably due to an increased stability of triazolyl urea linkage toward competing aqueous hydrolysis, thereby increasing the chances for amino oligonucleotide coupling to the plates. Other biological molecules with at least one reactive amino, thiol or hydroxyl group should experience higher sensitivity and higher loading under the present invention than attachments of biological molecules using the CDI method.

Having thus described the invention, it should be apparent that numerous modifications and adaptations may be resorted to without departing from the scope and fair meaning of the instant invention as set forth hereinabove and as described hereinbelow by the claims.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. §112.

What is claimed is:

1. A method of attaching a biological molecule having at least one reactive amino, thiol or hydroxyl group to a solid support having at least one available amino group, the method comprising the steps of:
(a) reacting the available amino group on the solid support with an activating compound, the activating compound having the structure:
L₁—X—L₂
wherein L₁ and L₂ are leaving groups, and X is a moiety capable of nucleophilic substitution so that the reaction results in L₁ being displaced by the available amino group on the solid support to form an activated support; and
(b) reacting the biological molecule with the activated support, thereby displacing L₂ and attaching the biological molecule to the solid support.

2. The method of claim 1 wherein L₁ and L₂ are independently selected from the group consisting of halogen, imidazole, triazole, pyrrole, pyrazole, thiazole, tetrazole and O-Aryl-R, and wherein R is selected from the group consisting of halogen, nitro, cyano, and alkoxy moiety.

3. The method of claim 2 wherein X is selected from the group consisting of: 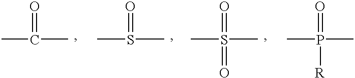
wherein R is selected from the group consisting of alkyl, aryl, and OR¹ having no greater than about 18 carbon atoms, and
wherein R¹ is selected from the group consisting of alkyl and aryl having no greater than about 18 carbon atoms.

4. The method of claim 1 wherein the activating compound is 1,2,4-carbonyl di triazole.

5. The method of claim 1 wherein step (b) comprises depositing between about 5 to about 25 nanoliters of the biological molecule in the circular spot having a diameter of between about 10 microns to about 500 microns at one or more sites on the activated support.

6. The method of claim 5 wherein the step of depositing comprises printing onto the activated solid support.

7. The method of claim 5 wherein in step b, the reaction occurs in a humid chamber.

8. The method of claim 6 wherein in step b, the reaction occurs in a humid chamber

9. The method of claim 1 wherein step (a) occurs in an organic solution.

10. The method of claim 9 wherein step (a) occurs in the presence of a tertiary organic base.

11. The method of claim 10 wherein step (b) occurs in an aqueous solution.

12. A method of attaching a biological molecule having at least one reactive amino, thiol or hydroxyl group to a solid support having at least one available amino group, the method comprising the steps of:
(a) reacting the available amino group on the solid support with an activating compound, the activating compound having the structure:
L₁—X—L₂
wherein L₁ and L₂ are identical leaving groups, and X is capable of nucleophilic substitution so that the reaction results in L₁ being displaced by the available amino group on the solid support to form an activated support; and
(b) reacting the biological molecule with the activated support, thereby displacing L₂ and attaching the biological molecule to the solid support.

13. The method of claim 12 wherein L₁ and L₂ are selected from the group consisting of halogen, imidazole, triazole, pyrrole pyrazole, thiazole, tetrazole and O-Aryl-R, and wherein R is selected from the group consisting of halogen, nitro, cyano, and alkoxy moiety.

14. The method of claim 13 wherein X is selected from the group consisting of: 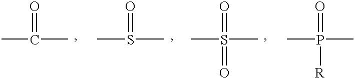
wherein R is selected from the group consisting of alkyl, aryl, and OR¹ having no greater than about 18 carbon atoms, and
wherein R¹ is selected from the group consisting of alkyl and aryl having no greater than about 18 carbon atoms.

15. The method of claim 12 wherein the activating compound is 1,2,4-carbonyl di triazole.

16. A solid-support attached to a biological molecule having the formula: 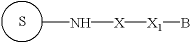
wherein S is the solid support,
wherein X is selected from the group consisting of: 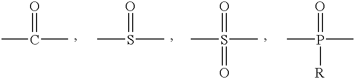
wherein R is selected from the group consisting of alkyl, aryl, and OR¹ having no greater than about 18 carbon atoms,
wherein R¹ is selected from the group consisting of alkyl and aryl having no greater than about 18 carbon atoms,
wherein X₁ is selected from the group consisting of NH, oxygen, and sulfur, and
wherein B is the biological molecule.

17. A solid-support of claim 16 having the formula:

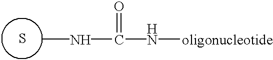

18. The method of claim 1 comprising the step of washing from the solid support non-bound compounds after step (a) and before step (b).

19. A method of attaching an organic molecule to a solid support, the method comprising the steps of:
(a) activating the solid support; and
(b) reacting the organic molecule with the activated support in a humid chamber, having a humidity of at least 60 percent relative humidity.
